SEQUENCE LISTING (20 sequences)

SEQ ID NO: 1 - DNA, 24 bp, Agrobacterium tumefaciens (rpoC)
tccaagatccatgaaacgacgcct

SEQ ID NO: 2 - DNA, 24 bp, Agrobacterium tumefaciens (rpoC)
ttggtcatttcctggttgcaggtg

SEQ ID NO: 3 - DNA, 24 bp, Deinococcus radiodurans (rpoC)
gtactacaccagccgtgagcgtat

SEQ ID NO: 4 - DNA, 24 bp, Deinococcus radiodurans (rpoC)
tctacgatacggcgttgttcgctg

SEQ ID NO: 5 - DNA, 24 bp, Mycobacterium tuberculosis (rpoC)
gtactacaccagccgtgagcgtat

SEQ ID NO: 6 - DNA, 24 bp, Mycobacterium tuberculosis (rpoC)
tctacgatacggcgttgttcgctg

SEQ ID NO: 7 - DNA, 24 bp, Shewanella oneidensis (rpoC)
gtactacaccagccgtgagcgtat

SEQ ID NO: 8 - DNA, 24 bp, Shewanella oneidensis (rpoC)
tctacgatacggcgttgttcgctg

SEQ ID NO: 9 - DNA, 20 bp, Shewanella oneidensis (udk)
gaccatcccaaagcgttaga

SEQ ID NO: 10 - DNA, 20 bp, Shewanella oneidensis (udk)
attgcaggaacataggacgg

SEQ ID NO: 11 - DNA, 24 bp, Vibrio cholera (rpoC)
ccaacggtcgtgtcaatcatcttg

SEQ ID NO: 12 - DNA, 24 bp, Vibrio cholera (rpoC)
aaggcgaaggtatgtacctgactg

SEQ ID NO: 13 - DNA, 28 bp, Artificial Sequence
  synthetic primer
aactatctctggtgcctctgtcggtatc

SEQ ID NO: 14 - DNA, 27 bp, Artificial Sequence
  synthetic primer
cgcacttgcccagatgtcgattacttt

SEQ ID NO: 15 - DNA, 20 bp, Artificial Sequence
  synthetic primer
gaccatcccaaagcgttaga

SEQ ID NO: 16 - DNA, 20 bp, Artificial Sequence
  synthetic primer
attgcaggaacataggacgg

SEQ ID NO: 17 - DNA, 23 bp, Artificial Sequence
  synthetic primer
  misc_feature 18, 19, 20, 21, 22, 23: n = A,T,C or G
gtttcccagtcacgatcnnnnnn

SEQ ID NO: 18 - DNA, 17 bp, Artificial Sequence
  synthetic primer
gtttcccagtcacgatc

SEQ ID NO: 19 - DNA, 23 bp, Artificial Sequence
  synthetic primer
  misc_feature 18, 19, 20, 21, 22, 23: n = A,T,C or G
gtttcccagtcacgatcnnnnnn

SEQ ID NO: 20 - DNA, 17 bp, Artificial Sequence
  synthetic primer
gtttcccagtcacgatc

(19) United States
(12) Patent Application Publication
(10) Pub. No.: US 2007/0264636 A1
(43) Pub. Date: Nov. 15, 2007

(54) CAPTURE AND RANDOM AMPLIFICATION PROTOCOL FOR IDENTIFICATION AND MONITORING OF MICROBIAL DIVERSITY

(75) Inventors: LAUREL DIANE CROSBY, Stanford, CA (US); CRAIG S. CRIDDLE, Cupertino, CA (US)

Correspondence Address: BOZICEVIC, FIELD & FRANCIS LLP, 1900 UNIVERSITY AVENUE, SUITE 200, EAST PALO ALTO, CA, 94303, US

(73) Assignee: The Board of Trustees of the Leland Stanford Junior University

(21) Appl. No.: 11/381,308

(22) Filed: May 2, 2006

Related U.S. Application Data
(60) Provisional application No. 60/677,120, filed on May 2, 2005.

(51) Int. Cl. C12Q 1/68

(52) U.S. Cl. 435/6

(57) ABSTRACT

Sequence capture and random amplification are used to generate a population of polynucleotides that reflect the sequence diversity of a starting microbial population. The CAPRA population is used in hybridization reactions for the assessment of diversity and for the quantitation of particular members in the starting population. The polynucleotide population can also be sequenced, and/or cloned for evaluation of sequence diversity, generation of probes, generation of microarrays comprising such sequences, and the like.

This invention was made with Government support under contract DE-FG03-00ER63046, and DE-FG02-04ER3763 awarded by the Department of Energy. The Government has certain rights in this invention.

### BACKGROUND OF THE INVENTION

An enormous amount of effort is being made worldwide by microbial ecologists to assess microbial diversity, and to identify microorganisms in environmental samples. Preferred methods for such assessment of diversity can also provide for identification and taxonomic classification. However, most bacteria from natural environments cannot be cultured with current techniques. In soil, estimates are that 80 to 99% of the microorganisms remain unidentified.

There are a variety of uses for this information, including the assessment of changes in microbial community structure that occur on temporal or spatial scales or that occur in response to environmental perturbations. For example, one use of microbial sequence analysis is identification of soil microorganisms useful in bioremediation. Another is in the analysis of infectious disease, and in assessment of public health issues.

A combination of retrieval of genetic sequences and polynucleotide probing of microbial samples can be used to detect specific sequences of uncultured bacteria in natural samples and to microscopically identify individual cells. Phylogenetic analysis of the retrieved sequence of an uncultured microorganism can reveal its closest culturable relatives and may, together with information on the physicochemical conditions of its natural habitat, facilitate more directed cultivation attempts.

For the analysis of complex communities such as multispecies biofilms and activated-sludge flocs, sets of probes specific for different taxonomic levels may be applied consecutively beginning with the more general and ending with the more specific (a hierarchical top-to-bottom approach), thereby generating increasingly precise information on the structure of the community. In situ growth rates and activities of individual cells may also be assessed.

The foundation of molecular microbial ecology lies in the ability to access and study the genetic information of microorganisms. The concept of using linear sequences of genetic information to tell organisms apart is based on the idea that the degree of relatedness of two organisms depends on the number of accumulated sequence differences. That is, organisms that have recently diverged tend to share more genetic information than organisms of more distant ancestry. Scoring these differences allows for the creation of detailed “family trees” that map out the relationships between all members of life on Earth.

The field of microbial ecology has seen great advances in the past decade, owing in part to the development of molecular methods for microbial community analysis. The advent of molecular tools has allowed for the exploration of the rich microbial diversity of the biosphere without the need to first culture the organisms of interest. Although ecological studies vary in scope from the identification of new and intriguing microorganisms to the complex interactions of mixed microbial communities, the ability to infer ecological significance depends on confidence in the molecular techniques used.

Combinations of 16S rRNA and PCR amplification have been the method of choice for many researchers. However, organisms with the least similar sequences are under-represented in PCR-based analyses compared to organisms with more similarity. Some organisms can have primer sequences that match as little as 65% of a universal 16S primer sequence. Under high stringency PCR, 16S rRNA sequences from these organisms would not be amplified. This problem with PCR primer specificity has lead to the development of degenerate mixtures of primer, where variable positions are included as the oligonucleotides are synthesized. This allows for amplification of sequences that differ at various points in the priming sequences, but there is a limit to the amount of degeneracy that can be incorporated into a primer set.

Traditional PCR makes use of defined priming sequences to initiate synthesis of new strands. This requires that all intended targets have the correct priming sequences, a condition which is not possible on a universal scale with either the 16S rRNA genes or protein-encoding genes. If genes from different organisms are amplified at different rates, the products of PCR amplification will not reflect the initial ratios. Cycle number also plays a role in the final product ratios, because of the plateau in synthesis observed at higher cycles. If population densities differ by several orders of magnitude, some populations may reach the plateau phase earlier than others, skewing the ratios in favor of those that were initially least abundant. In other cases, all populations may reach the plateau phase and the gene products from different organisms will appear as a final ratio of one to one. In addition to variations in priming sites for PCR, there are other biases that are related to the amplification of mixed templates. Random variations in template amplification during early cycles are exacerbated in the product ratios at later cycles, a concept known as “drift”. Another problem with amplification of mixed templates relates to template-template interactions, particularly among homologous genes. For pure cultures the amplified products represent identical copies of the same original target. However, in mixed cultures, these different templates can hybridize to each other, forming chimeras and heteroduplexes.

A preferred tool for molecular microbial community analysis will have several fundamental characteristics. It should be universally applicable to all different groups in the prokaryotic domain and should have a level of resolution that is sufficient to tell the different organisms apart. The technique should be accurate in representing the true composition of the environment, and sensitive enough to identify populations that are in low abundance. In addition, one of the most desirable features is to have a technique that quantitatively reflects the ratios of different organisms in a sample. The ability to capture and sequence genetic markers is also of interest. The present invention addresses this need.

### SUMMARY OF THE INVENTION

Methods are provided for the assessment of microbial diversity, and for the identification of genes through sequence capture and random amplification to generate a population of polynucleotides that reflect the sequence diversity of the starting microbial population. The polynucleotide population may be used in hybridization reactions for the assessment of diversity and for the quantitation of particular members in the starting population. The polynucleotide population may also be sequenced, and/or cloned for evaluation of sequence diversity, generation of probes, and the like.

Genomic DNA is extracted from environmental samples, clinical samples, pure cultures, etc. and sheared into fragments. Sheared polynucleotides are then incubated with single stranded DNA probes complementary to a sequence of interest under stringency conditions that permit retention of the desired sequences. The hybrids thus formed are recovered, e.g. by column, paramagnetic particle, electrophoresis, or binding to any other substrate, etc. The captured polynucleotides are amplified with random primers, e.g. fully degenerate random hexamer primers, or priming sequences that include a fully degenerate segment of nucleotides at the 3′ end. The resulting CAPRA (capture and random amplification) polynucleotides are used in diversity assessment, monitoring, sequencing, cloning, and the like. As shown herein, CAPRA-based analyses are sensitive, accurate, and quantitative in evaluation of pure cultures, model communities, and unknown environmental samples.

In one embodiment of the invention, the CAPRA polynucleotide population is cloned into suitable vectors for sequencing, or is directly sequenced. The sequencing results can be used to identify novel genes recovered during the bead capture and random amplification technique. The polynucleotides can also be printed onto DNA microarrays for various downstream analytic applications.

In another embodiment of the invention, the sequence information from CAPRA polynucleotide populations is retained in a database, for identification of unknown microbes, comparison of populations over spatial and temporal distance, response to stresses, etc.

### BRIEF DESCRIPTION OF THE DRAWINGS

FIG. 1: Gene coverage of captured fragments for S. oneidensis

FIG. 2: Frequency of clones representing a given locus

FIG. 3: Overview of Gene Capture and Random Amplification (CAPRA). Genomic DNA samples are sheared into fragments, with some fragments containing the conserved capture sequence (circles.) Genes are captured at these sites using an oligonucleotide probe, whereas fragments lacking the capture site are eliminated as background. The pool of captured fragments are subsequently amplified using a random PCR protocol, and amplified products can be cloned and sequenced, or screened according to unique adjacent sequences (diamonds.) In this example, closed diamonds represent fragments that are successfully captured and amplified, while the open diamonds represent detection sites that are lost with background. Although some detection sites may be lost, all populations of homologous genes in the mixed sample should be equally affected. Given that genes are captured at the conserved site with equal efficiency and amplified without bias, this allows for quantitative preservation of the ratios of target genes.

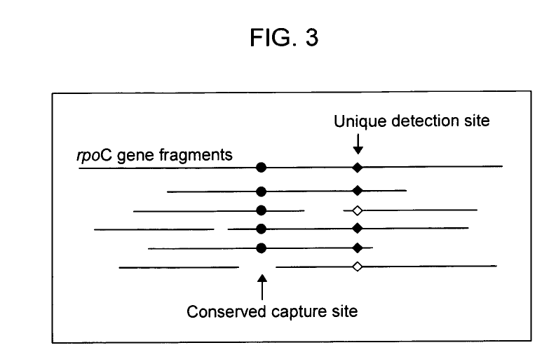

FIG. 4: DNA samples prepared for gene capture and random amplification. DNA is extracted from cells (lane a) and sheared into fragments using a GeneMachines Hydroshear apparatus at speed code 12 for a fragment size range of 2-5 kb (lane b). Captured fragments from this DNA pool are then randomly amplified (lane c), producing a further truncated set of fragments of approximately 500-1200 base pairs.

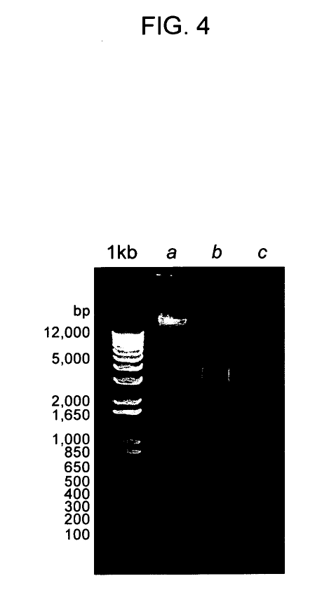

FIG. 5: Gene capture and random amplification of non-homologous genes, rpoC and udk from a pure culture of S. oneidensis. Aliquots were sacrificed over successive rounds of random amplification and genes measured using quantitative PCR. Gene capture reflects an increase in the signal:noise ratio of rpoC:udk by over 300 times, and both genes are amplified non-specifically over four orders of magnitude at equivalent rates.

FIGS. 6A-6B: Gene capture and random amplification with a bead-based DNA capture probe targeting the rpoC gene. Initial ratios of a mixture of 5 different organisms were measured by quantitative PCR and compared to final ratios after (a) gene capture, and (b) gene capture plus random amplification, with V. cholerae as the internal standard. The solid line with slope 1 represents 100% recovery relative to the standard, and the dashed lines represent recovery by a factor of two above and below the expected values. For gene capture (a), each sample was sacrificed and measured for each of the 5 members. For the full gene capture and random amplification assay (b), points represent one captured sample and 3 replicates of the random amplification reaction with error bars representing the 95% confidence interval. (D. radiodurans and M. tuberculosis were also present in the mixture, but not quantified.)

FIGS. 7A-7B. DNA tagging reaction using random hexamers with a specific 5′ end. The tagging reaction requires 2 rounds of synthesis in order to generate at least one strand with a complementary sequence.

### DEFINITIONS

The term “oligomer” is used herein to indicate a chemical entity that contains a plurality of monomers. As used herein, the terms “oligomer” and “polymer” are used interchangeably. Examples of oligomers and polymers include polydeoxyribonucleotides (DNA), polyribonucleotides (RNA), other nucleic acids that are C-glycosides of a purine or pyrimidine base, polypeptides (proteins) or polysaccharides (starches, or polysugars), as well as other chemical entities that contain repeating units of like chemical structure.

The term “nucleic acid” as used herein means a polymer composed of nucleotides, e.g., deoxyribonucleotides or ribonucleotides, or compounds produced synthetically (e.g., PNA as described in U.S. Pat. No. 5,948,902 and the references cited therein) which can hybridize with naturally occurring nucleic acids in a sequence specific manner analogous to that of two naturally occurring nucleic acids, e.g., can participate in Watson-Crick base pairing interactions.

The terms “ribonucleic acid” and “RNA” as used herein mean a polymer composed of ribonucleotides. The terms “deoxyribonucleic acid” and “DNA” as used herein mean a polymer composed of deoxyribonucleotides. The term “oligonucleotide” as used herein denotes single stranded nucleotide multimers of from about 10 to 100 nucleotides and up to 200 nucleotides in length.

The term “sample” as used herein relates to a material or mixture of materials, typically, although not necessarily, in fluid form, containing one or more components of interest.

The terms “nucleoside” and “nucleotide” are intended to include those moieties that contain not only the known purine and pyrimidine bases, but also other heterocyclic bases that have been modified. Such modifications include methylated purines or pyrimidines, acylated purines or pyrimidines, alkylated riboses or other heterocycles. In addition, the terms “nucleoside” and “nucleotide” include those moieties that contain not only conventional ribose and deoxyribose sugars, but other sugars as well. Modified nucleosides or nucleotides also include modifications on the sugar moiety, e.g., wherein one or more of the hydroxyl groups are replaced with halogen atoms or aliphatic groups, or are functionalized as ethers, amines, or the like.

The term “stringent assay conditions” as used herein refers to conditions that are compatible to produce binding pairs of nucleic acids, e.g., surface bound and solution phase nucleic acids, of sufficient complementarity to provide for the desired level of specificity in the assay while being less compatible to the formation of binding pairs between binding members of insufficient complementarity to provide for the desired specificity. Stringent assay conditions are the summation or combination (totality) of both hybridization and wash conditions.

Stringent assay conditions are hybridization conditions that are at least as stringent as the above representative conditions, where a given set of conditions are considered to be at least as stringent if substantially no additional binding complexes that lack sufficient complementarity to provide for the desired specificity are produced in the given set of conditions as compared to the above specific conditions, where by “substantially no more” is meant less than about 5-fold more, typically less than about 3-fold more. Other stringent hybridization conditions are known in the art and may also be employed, as appropriate.

Low stringency hybridization conditions in the context of nucleic acid hybridization (e.g., as in array, Southern or Northern hybridizations) are sequence dependent, and are different under different experimental parameters. The specific temperature and salt concentrations for the reaction may be tailored to capture the sequences of interest. An example of low stringency conditions includes hybridization in a buffer comprising 5×SSC and 1% SDS at from about 20 to about 42° C., with a wash of 0.2×SSC and 0.1% SDS at from about 20 to about 42° C.

A “stringent hybridization” and “stringent hybridization wash conditions” in the context of nucleic acid hybridization (e.g., as in array, Southern or Northern hybridizations) are sequence dependent, and are different under different experimental parameters. Stringent hybridization conditions that can be used to identify nucleic acids within the scope of the invention can include, e.g., hybridization in a buffer comprising 50% formamide, 5×SSC, and 1% SDS at 42° C., or hybridization in a buffer comprising 5×SSC and 1% SDS at 65° C., both with a wash of 0.2×SSC and 0.1% SDS at 65° C. Exemplary stringent hybridization conditions can also include a hybridization in a buffer of 40% formamide, 1 M NaCl, and 1% SDS at 37° C., and a wash in 1×SSC at 45° C. Yet additional stringent hybridization conditions include hybridization at 60° C. or higher and 3×SSC (450 mM sodium chloride/45 mM sodium citrate) or incubation at 42° C. in a solution containing 30% formamide, 1M NaCl, 0.5% sodium sarcosine, 50 mM MES, pH 6.5. Those of ordinary skill will readily recognize that alternative but comparable hybridization and wash conditions can be utilized to provide conditions of similar stringency.

The phrase “oligonucleotide bound to a surface of a solid support” refers to an oligonucleotide or mimetic thereof that is immobilized on a surface of a solid substrate in a feature or spot, where the substrate can have a variety of configurations, e.g., a sheet, bead, or other structure. In certain embodiments, the collections of features of oligonucleotides employed herein are present on a surface of the same planar support, e.g., in the form of an array.

The term “array” encompasses the term “microarray” and refers to an ordered array presented for binding to nucleic acids and the like. Arrays are generally made up of a plurality of distinct or different features. The term “feature”¹ is used interchangeably herein with the terms: “features,” “feature elements,” “spots,” “addressable regions,” “regions of different moieties,” “surface or substrate immobilized elements” and “array elements,” where each feature is made up of oligonucleotides bound to a surface of a solid support, also referred to as substrate immobilized nucleic acids.

An “array,” includes any one-dimensional, two-dimensional or substantially two-dimensional (as well as a three-dimensional) arrangement of addressable regions (i.e., features, e.g., in the form of spots) bearing nucleic acids, particularly oligonucleotides or synthetic mimetics thereof (i.e., the oligonucleotides defined above), and the like. Where the arrays are arrays of nucleic acids, the nucleic acids may be adsorbed, physisorbed, chemisorbed, or covalently attached to the arrays at any point or points along the nucleic acid chain.

A typical array may contain one or more, including more than two, more than ten, more than one hundred, more than one thousand, more ten thousand features, or even more than one hundred thousand features, in an area of less than 20 cm² or even less than 10 cm², e.g., less than about 5 cm², including less than about 1 cm², less than about 1 mm², e.g., 100μ², or even smaller. For example, features may have widths (that is, diameter, for a round spot) in the range from a 10 μm to 1.0 cm. In other embodiments each feature may have a width in the range of 1.0 μm to 1.0 mm, usually 5.0 μm to 500 μm, and more usually 10 μm to 200 μm. Non-round features may have area ranges equivalent to that of circular features with the foregoing width (diameter) ranges. At least some, or all, of the features are of different compositions (for example, when any repeats of each feature composition are excluded the remaining features may account for at least 5%, 10%, 20%, 50%, 95%, 99% or 100% of the total number of features). Inter-feature areas will typically (but not essentially) be present which do not carry any nucleic acids (or other biopolymer or chemical moiety of a type of which the features are composed). Such inter-feature areas typically will be present where the arrays are formed by processes involving drop deposition of reagents but may not be present when, for example, photolithographic array fabrication processes are used. It will be appreciated though, that the inter-feature areas, when present, could be of various sizes and configurations.

Each array may cover an area of less than 200 cm², or even less than 50 cm², 5 cm², 1 cm², 0.5 cm², or 0.1 cm². In certain embodiments, the substrate carrying the one or more arrays will be shaped generally as a rectangular solid (although other shapes are possible), having a length of more than 4 mm and less than 150 mm, usually more than 4 mm and less than 80 mm, more usually less than 20 mm; a width of more than 4 mm and less than 150 mm, usually less than 80 mm and more usually less than 20 mm; and a thickness of more than 0.01 mm and less than 5.0 mm, usually more than 0.1 mm and less than 2 mm and more usually more than 0.2 and less than 1.5 mm, such as more than about 0.8 mm and less than about 1.2 mm. With arrays that are read by detecting fluorescence, the substrate may be of a material that emits low fluorescence upon illumination with the excitation light. Additionally in this situation, the substrate may be relatively transparent to reduce the absorption of the incident illuminating laser light and subsequent heating if the focused laser beam travels too slowly over a region. For example, the substrate may transmit at least 20%, or 50% (or even at least 70%, 90%, or 95%), of the illuminating light incident on the front as may be measured across the entire integrated spectrum of such illuminating light or alternatively at 532 nm or 633 nm.

Arrays can be fabricated using drop deposition from pulse-jets of either nucleic acid precursor units (such as monomers) in the case of in situ fabrication, or the previously obtained nucleic acid. Such methods are described in detail in, for example, the previously cited references including U.S. Pat. No. 6,242,266, U.S. Pat. No. 6,232,072, U.S. Pat. No. 6,180,351, U.S. Pat. No. 6,171,797, U.S. Pat. No. 6,323,043, U.S. patent application Ser. No. 09/302,898 filed Apr. 30, 1999 by Caren et al., and the references cited therein. As already mentioned, these references are incorporated herein by reference. Other drop deposition methods can be used for fabrication, as previously described herein. Also, instead of drop deposition methods, photolithographic array fabrication methods may be used. Inter-feature areas need not be present particularly when the arrays are made by photolithographic methods as described in those patents.

### DETAILED DESCRIPTION OF THE EMBODIMENTS

Before the subject invention is described further, it is to be understood that the invention is not limited to the particular embodiments of the invention described below, as variations of the particular embodiments may be made and still fall within the scope of the appended claims. It is also to be understood that the terminology employed is for the purpose of describing particular embodiments, and is not intended to be limiting. Instead, the scope of the present invention will be established by the appended claims.

In this specification and the appended claims, the singular forms “a,” “an” and “the” include plural reference unless the context clearly dictates otherwise. Unless defined otherwise, all technical and scientific terms used herein have the same meaning as commonly understood to one of ordinary skill in the art to which this invention belongs.

Where a range of values is provided, it is understood that each intervening value, to the tenth of the unit of the lower limit unless the context clearly dictates otherwise, between the upper and lower limit of that range, and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, the preferred methods, devices and materials are now described.

All publications mentioned herein are incorporated herein by reference for the purpose of describing and disclosing the invention components that are described in the publications that might be used in connection with the presently described invention.

Methods are provided for the assessment of microbial diversity, and for the identification of genes through sequence capture and random amplification to generate a population of polynucleotides that reflect the sequence diversity of the starting microbial population. Genomic DNA is extracted and sheared into fragments. The sheared polynucleotides are then incubated with single stranded DNA probes complementary to a sequence of interest under stringency conditions that permit retention of the desired genus of sequences. The hybrids thus formed are recovered, e.g. by column, paramagnetic particle, gel electrophoresis, etc. The captured polynucleotides are amplified with random primers, e.g. fully degenerate random hexamer primers. The resulting CAPRA (capture and random amplification) polynucleotides are used in diversity assessment, monitoring, sequencing, cloning, and the like. As shown herein, CAPRA-based analyses are sensitive, accurate, and quantitative in evaluation of pure cultures, model communities, and unknown environmental.

The capture and random amplification technique is well suited to any gene for which a suitable capture probe can be identified. This ability to recover genes from the environment also leads to possibilities in monitoring other types of genetic material, such as that encoded by viruses and bacteriophage.

The methods of the invention provide certain advantages over conventional methods. Traditional PCR requires the use of two known primers in order to amplify a desired sequence. In mixed cultures, however, priming sites can differ between target populations. PCR primer bias prevents current techniques from being quantitative in describing microbial populations. Traditional PCR is also limited by primer design. For targeting genes in mixed communities, degenerate primers are often used to amplify target sequences that have slight design heterogeneity, but substantial degeneracy in primer design leads to hybridization of primers to each other, rather than the intended target.

### Microbial Sample

Test samples of microbes include microbe communities and isolated strains. Samples of interest include environmental samples, e.g. ground water, sea water, mining waste, etc.; biological samples, e.g. lysates prepared from crops, tissue samples, etc.; manufacturing samples, e.g. time course during preparation of pharmaceuticals; and the like.

It will be understood by those of skill in the art that there are many sources of microbial communities, including biofilm communities, e.g. in air filtration systems, in tissue samples from implanted medical devices.

Soil organisms are of interest for many assays, including bioremediation, landfill remediation, crop areas, etc.

Samples are usually provided in a suspension or solution, and may contain as few as a single microbial cell, usually at least about 10², more usually at least about 10³, 10⁴, 10⁵ or more cells, and may contain from one, two, three, four, to tens, hundreds or more of different species.

The term genome refers to all nucleic acid sequences (coding and non-coding) and elements present in or originating from any virus, single cell (prokaryote and eukaryote) or each cell type and their organelles (e.g. mitochondria) in an organism. The term genome also applies to any naturally occurring or induced variation of these sequences that may be present in a mutant or disease variant of any virus or cell type. These sequences include, but are not limited to, those involved in the maintenance, replication, segregation, and higher order structures (e.g. folding and compaction of DNA in chromatin and chromosomes), or other functions, if any, of the nucleic acids as well as all the coding regions and their corresponding regulatory elements needed to produce and maintain each particle, cell or cell type in a given organism.

The genomic source may be prepared using any convenient protocol. In many embodiments, the genomic source is prepared by first obtaining a starting composition of genomic DNA, e.g., a nuclear fraction of a cell lysate, where any convenient means for obtaining such a fraction may be employed and numerous protocols for doing so are well known in the art, e.g. detergent lysis, French press, freeze thaw, etc. The genomic source is, in many embodiments of interest, genomic DNA representing the entire genome from a particular organism, or community.

Where desired, the genomic source may be fragmented or sheared in the generation protocol, as desired, to produce a fragmented genomic source, where the molecules have a desired average size range, e.g., up to about 10 Kb, such as up to about 1 Kb, where fragmentation may be achieved using any convenient protocol, including but not limited to: mechanical protocols, e.g., sonication, shearing, etc., chemical protocols, e.g., enzyme digestion, etc. The size range of sheared DNA fragments may be modified to influence the detection sensitivity in downstream applications.

Following provision of the initial genomic source, and any initial processing steps (e.g., fragmentation, amplification, etc.) as described above, the collection of solution phase nucleic acids is prepared for use in the subject methods. Typically the collection of solution phase nucleic acids prepared from the initial genomic source is one that has substantially the same complexity as the complexity of the initial genomic source. Complexity, as used in describing the product nucleic acid collection/population, refers to the number of distinct or different nucleic acid sequences found in a collection of nucleic acids relative to the number of distinct or different nucleic acid sequences found in the genomic source.

### Capture Methods

The capture probe is complementary to a target nucleic acid sequence present in the microbial population being studied. Preferred targets are housekeeping genes, e.g. those involved in nucleic acid metabolism (gmk, guanylate kinase; uridine kinase, dihydrofolate reductase [DHFR], DNA polymerase, RNA polymerase; adenylate kinase gene (adk), RecA gene (recA)), glucose metabolism (tpi, triosephosphate isomerase), protein metabolism (the 16S rRNA gene; hsp-60, heat-shock protein 60), and the like. In one embodiment of the invention the target sequence is rpoC. Functional genes may also be preferred, e.g. when information about the diversity or distribution of a given gene is desired, e.g. ammonia monooxygenase (amoA), nitrite reductase (nirK, nirS), etc. Preferred target sites include regions of low variability, e.g. active site regions, etc. The high number of completely sequenced microbial genomes allows for ready analysis and selection of suitable target sequences.

The capture probe may comprise a single sequence complementary to a selected target, or may comprise a cocktail of sequences, where degeneracy is introduced in order to permit for greater capture. Frequently such cocktails will comprise third position degeneracy, and may include known variations in sequence among the target microbial community. The capture probe is usually a single stranded oligonucleotide of at least about 12 nucleotides in length, more usually at least about 15 nucleotides in length, and may be at least about 18 nucleotides, at least about 25 nucleotides, at least about 100 nucleotides, and usually not more than about 10³ nucleotides in length.

For hybridization probes, it may be desirable to use nucleic acid analogs, in order to improve the stability and binding affinity. A number of modifications have been described that alter the chemistry of the phosphodiester backbone, sugars or heterocyclic bases. Nucleic acid probes may be used to identify expression of the gene in a biological specimen. The manner in which one probes cells for the presence of particular nucleotide sequences, as genomic DNA or RNA, is well-established in the literature and does not require elaboration here.

The capture probe is typically bound to a substrate, for separation of bound target to capture probe. Such substrates include solid surfaces, such as a microwell plate, dish, and the like; to resins, e.g. column resins; and the like.

In one embodiment of the invention, the capture oligonucleotides are coupled to a magnetic reagent, such as a superparamagnetic microparticle (microparticle). Herein incorporated by reference, Molday (U.S. Pat. No. 4,452,773) describes the preparation of magnetic iron-dextran microparticles and provides a summary describing the various means of preparing particles suitable for attachment to biological materials. A description of polymeric coatings for magnetic particles used in high gradient magnetic separation (HGMS) methods are found in DE 3720844 (Miltenyi) and U.S. Pat. No. 5,385,707. Methods to prepare superparamagnetic particles are described in U.S. Pat. No. 4,770,183. The microparticles will usually be less than about 100 nm in diameter, and usually will be greater than about 10 nm in diameter.

The exact method for coupling is not critical to the practice of the invention, and a number of alternatives are known in the art. Direct coupling attaches the capture oligonucleotides to the particles. Indirect coupling can be accomplished by several methods. The oligonucleotides may be coupled to one member of a high affinity binding system, e.g. biotin, and the particles attached to the other member, e.g. avidin. Indirect coupling methods allow the use of a single magnetically coupled entity, e.g. avidin, etc., with a variety of capture oligonucleotides.

The sheared genomic DNA is denatured, and contacted with the capture probe under conditions that permit hybridization to the target gene in a variety of organisms while not retaining unrelated DNA. The hybridization and wash conditions are usually selected so as to retain sequences that have at least about 50% sequence identity; at least about 60% sequence identity; at least about 75% sequence identity; at least about 85% sequence identity; at least about 90% sequence identity; or more. Such conditions are readily determined by one of skill in the art using empirical and theoretical methods. The hybridization may be performed before or after coupling of the probe to the paramagnetic particle.

The substrate bound to the capture probe and captured DNA is then washed free of non-specifically attached polynucleotides. Where the substrate is a paramagnetic particle, the suspension is applied to a separation device. Exemplary magnetic separation devices are described in WO/90/07380, PCT/US96/00953 and EP 438,520, herein incorporated by reference. The matrix for separation should have adequate surface area to create sufficient magnetic field gradients in the separation device to permit efficient retention of magnetically labeled particles. The volume necessary for a given separation may be empirically determined, and will vary with the size, density, affinity, etc. The flow rate will be determined by the size of the column, but will generally not require a cannula or valve to regulate the flow.

The paramagnetic particles are retained in the magnetic separation device in the presence of a magnetic field, usually at least about 100 mT, more usually at least about 500 mT, usually not more than about 2 T, more usually not more than about 1 T. The source of the magnetic field may be a permanent or electromagnet. After the initial binding, the device may be washed with any suitable physiological buffer to remove unbound material.

Where greater purity is desired, additional separation steps may be performed. The eluted, magnetic fraction may be passed over a second magnetic column to reduce the non-specifically bound material.

### Random Amplification

The captured polynucleotide pool in then randomly amplified. The term “amplify” in reference to a polynucleotide means to use any method to produce multiple copies of a polynucleotide segment, called the “amplicon” or “amplification product”, by replicating a sequence element from the polynucleotide or by deriving a second polynucleotide from the first polynucleotide and replicating a sequence element from the second polynucleotide. The copies of the amplicon may exist as separate polynucleotides or one polynucleotide may comprise several copies of the amplicon. The precise usage of amplify is clear from the context to one skilled in the art.

A preferred amplification method utilizes PCR (see Saiki et al. (1988) Science 239:487-4391). Random amplification may be performed in a variety of ways, such as rolling circle amplification with random hexanucleotide primers and the Phi29 DNA polymerase, or equivalent.

Random amplification may involve the use of a variety of thermostable DNA polymerases with and without exonuclease activities. Polymerases containing the 5′ to 3′ strand-displacement activity are important for generating long amplicons during the tagging step. However, using polymerases that lack the 5′ to 3′ strand displacement activity may be useful in limiting the processivity of the polymerase, and therefore limiting the size of the amplicons for microarray hybridization. Polymerases may also be used that have 3′ to 5′ exonuclease activities. This may be useful for degrading the terminal priming sites after the amplification cycles are complete, thus eliminating the risk of forming hairpin structures.

Another variation of random amplification may be used to amplify RNA, such as rRNA, tRNA, or mRNA fragments that are selectively enriched by hybridization with a capture oligonucleotide. In this embodiment, random amplification protocol may be modified with use of a reverse transcriptase enzyme or a DNA polymerase that also contains a reverse transcriptase activity. This may be important in determining not only the presence of an organism, but the functional activity of an organism in the environment. The presence and activity of any given organism may be compared and contrasted by amplifying DNA and mRNA in two separate reactions with two separate, but independently identifiable labels. Both labels may be competitively hybridized directly to a microarray, in a manner equivalent to the red-green mRNA expression assays.

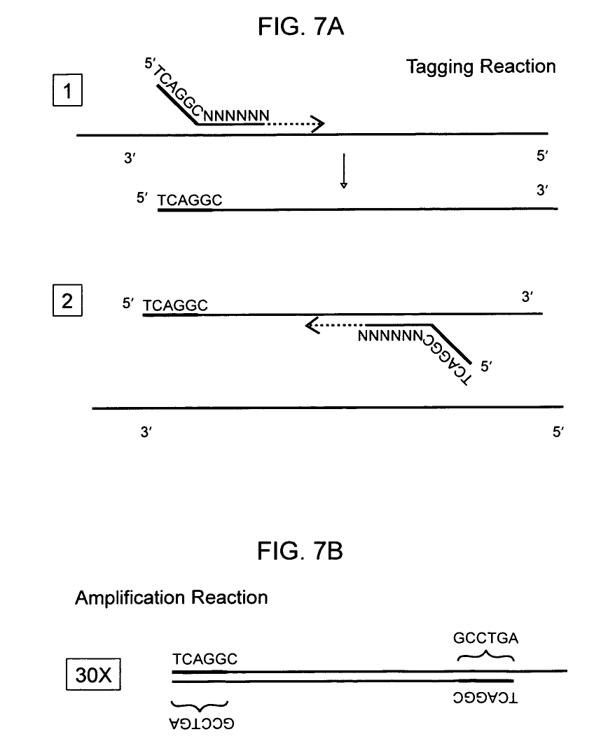

In another embodiment, random amplification utilizes a two-step process, where the first step, tagging, allows for the incorporation of a new priming site into subsequent fragments, and the second step involves amplification from the newly-incorporated priming sequence, or tag (see Bohlander et al. (1992) Genomics. 1992 August; 13(4):1322-4.) In such methods, a first step utilizes a pool of primers, each of which comprises two regions of sequence (initial primer). The region at the 3′ end of the primer is fully degenerate. The fully degenerate sequence may be at least up to 6 nucleotides in length, although longer regions may be used. The preferred pool of primers is completely degenerate, i.e. containing all possible combinations of bases in each of the last 6 positions. The 5′ region comprises a defined sequence of sufficient length to serve as a new priming site during the second step. The 5′ region may be between 6 and 30 nucleotides long, or more. A schematic of the process is shown in FIG. 7. The defined region may be designed to include sequences for restriction enzyme cutting sites, regions of varying thermal hybridization efficiency, or other characteristics that make the subsequent amplicons useful for downstream applications (e.g. multiplexing different random amplification reactions in the same tube or different tubes, using restriction enzymes to recover cloned inserts, etc.)

In a first round of amplification with the initial primer, polymerization commences from the random region. The defined sequences of the 5′ region are incorporated at the terminal ends of each new amplicon. Thus, the initial primer serves to “tag” a fragment of DNA with a new and defined priming sequence. A second round of priming and extension with the initial primer serves to generate the complement of the initial primer sequence. Multiple rounds of amplification may be used, but no less than two, in order to incorporate the defined region at both ends. More cycles, e.g. about 5, about 10, about 15, about 30 or more, serve to increase the amount of template for the second step. The intervening sequences between the two priming sites corresponds to DNA sequences that were originally present in the sample.

The second step of the 2-step random amplification protocol is a conventional PCR reaction. The “amplification primers” have the sequence of the 5′ region of the initial primer, but lack the degenerate 3′ region. Amplification with the amplification primers results in exponential increase in the number of DNA template strands.

A modification of previously reported methods of 2-step random amplification utilizes an initial amplification step with a thermophilic polymerase under identical reaction conditions as the amplification reactions utilizing the amplification primers. Defined primer sequences, with and without the fully degenerate random hexamer on the 3′ end, can be mixed in the same tube before or after the tagging step. Alternatively, the amplification primers are added after the tagging step.

The cycling conditions in both the tagging and amplification steps may be modified to influence the size and abundance of the subsequent amplicons. For example, long annealing and extension times allow the polymerase to generate long DNA polymers, whereas shorter annealing and extension times limit the capabilities of the polymerase. The size of the amplified fragments is important in cloning, where long fragments are preferred. On the other hand, hybridization of randomly amplified fragments to DNA microarrays requires short fragments, and cycling conditions can be used to generate variably-sized fragments, depending on the desires of the operator.

Random amplification using the 2-step protocol may also be used as a means to label fragments for subsequent hybridization to DNA microarrays. A variety of fluorescent molecules, nanoparticles, or other labeling chemistries may be used to label fragments. These labels are incorporated into each new DNA strand.

Excess labeled primer may also be used to bind to complementary sequences after amplification cycles are complete. Because each single strand of DNA contains the priming sequence and its complement on opposite side of the fragment, the single-strand of DNA, especially short fragments, may have a propensity to fold back on itself and form a hairpin structure. This could limit the binding capacity to a DNA microarray, since the DNA strand may preferentially bind to itself instead of the microarray target. Thus, the excess label would have the effect of binding to the complementary target, limiting the formation of hairpin structures. A beneficial secondary effect is that each DNA strand contains two fluorescent labels, with one at each end. This may be especially useful when using gold nanoparticles as labels, since gold nanoparticles have unique characteristics when two or more particles come into close proximity.

In the various methods of random amplification, typically an excess of random primers is employed, such that in a given primer set employed in the subject invention, multiple copies of each different random primer sequence is present, and the total number of primer molecules in the set far exceeds the total number of distinct primer sequences, where the total number may range from about 1.0×10¹⁰ to about 1.0×10²⁰, such as from about 1.0×10¹³ to about 1.0×10¹⁷, e.g., 3.7×10¹⁵. The primers described above and throughout this specification may be prepared using any suitable method, such as, for example, the known phosphotriester and phosphite triester methods, or automated embodiments thereof. In one such automated embodiment, dialkyl phosphoramidites are used as starting materials and may be synthesized as described by Beaucage et al. (1981), Tetrahedron Letters 22, 1859. One method for synthesizing oligonucleotides on a modified solid support is described in U.S. Pat. No. 4,458,066.

The primers are mixed with a solution containing the target DNA (the template), a thermostable DNA polymerase and deoxynucleoside triphosphates (dNTPS) for all four deoxynucleotides. The mix is then heated to a temperature sufficient to separate the two complementary strands of DNA. The mix is next cooled to a temperature sufficient to allow the primers to specifically anneal to sequences flanking the gene or sequence of interest. The temperature of the reaction mixture is then optionally reset to the optimum for the thermostable DNA polymerase to allow DNA synthesis (extension) to proceed. The temperature regimen is then repeated to constitute each amplification cycle. Thus, PCR consists of multiple cycles of DNA melting, annealing and extension.

The PCR methods used in the methods of the present invention are carried out using standard methods (see, e.g., McPherson et al., PCR (Basics: From Background to Bench) (2000) Springer Verlag; Dieffenbach and Dveksler (eds) PCR Primer: A Laboratory Manual (1995) Cold Spring Harbor Laboratory Press; Erlich, PCR Technology, Stockton Press, New York, 1989; Innis et al., PCR Protocols: A Guide to Methods and Applications, Academic Press, Harcourt Brace Javanovich, N.Y., 1990; Barnes, W. M. (1994) Proc Nat Acad Sci USA, 91, 2216-2220). The primers and oligonucleotides used in the methods of the present invention are preferably DNA and analogs thereof, e.g. phosphorothioates; phosphorodithioates, where both of the non-bridging oxygens are substituted with sulfur; phosphoroamidites; alkyl phosphotriesters and boranophosphates. Achiral phosphate derivatives include 3′-O′-5′-S-phosphorothioate, 3′-S-5′-O-phosphorothioate, 3′-CH₂-5′-O-phosphonate and 3′—NH-5′-O-phosphoroamidate. Such nucleic acids can be synthesized using standard techniques

The number of cycles of amplification will generate sufficient polynucleotide product to analyze an aliquot for the desired purpose. Typically at least about 10 cycles, at least about 15 cycles, at least about 20 cycles, at least about 30 cycles or more will be utilized. The number of cycles for a particular application will be determined by the amount of initial template present, the requirements for the desired use, and the like.

### Analysis and Identification

The CAPRA polynucleotide composition may be used in a variety of methods for microbial identification, sequence analysis and the like. In some embodiments, the CAPRA polynucleotide composition is hybridized to known sequences, e.g. to a polynucleotide array, to assess the complexity of the population. In such experiments, the presence of specific sequences is correlated with the source organism, and may include quantitative assessment of the population.

In other methods, the CAPRA polynucleotide composition is sequenced, either directly or following cloning into a suitable vector, in order to obtain further information about the microbial diversity in the targeted population. Such sequence information may be stored in a database, used to direct the assembly of a polynucleotide array, and the like. Methods of cloning and sequences target sequences of interest are known to those of skill in the art. Where hybridization is being performed, the CAPRA polynucleotide composition may be labeled prior to contacting with an array, filter, etc. Using the above protocols, at least a first collection of nucleic acids is produced from the genomic source. Optionally, competitive hybridizations are performed, e.g. with a control source which may be a known collection, a time series from a specific environment, and the like. The populations may be labeled with the same or different labels. The constituent members of the above produced collections typically range in length from about 100 to about 10,000 nt, such as from about 200 to about 10,000 nt, including from about 100 to 1,000 nt, from about 100 to about 500 nt, etc.

The population(s) of labeled nucleic acids produced by the subject methods are contacted to a plurality of different surface immobilized elements (i.e., features on an array) under conditions such that nucleic acid hybridization to the surface immobilized elements can occur. The collections can be contacted to the surface immobilized elements either simultaneously or serially. In many embodiments the compositions are contacted with the plurality of surface immobilized elements, e.g., the array of distinct oligonucleotides of different sequence, simultaneously. Depending on how the collections or populations are labeled, the collections or populations may be contacted with the same array or different arrays, where when the collections or populations are contacted with different arrays, the different arrays are substantially, if not completely, identical to each other in terms of feature content and organization.

Typically the substrate immobilized nucleic acids that make up the features of the arrays employed in the subject methods are oligonucleotides, although in some instances longer probes may be used. By oligonucleotide is meant a nucleic acid having a length ranging from about 10 to about 200 nt including from about 10 or about 20 nt to about 100 nt, where in many embodiments the immobilized nucleic acids range in length from about 50 to about 90 nt or about 50 to about 80 nt, such as from about 50 to about 70 nt.

The oligonucleotides that make up the distinct features are ones that have been designed according to one or more particular parameters to be suitable for use in a given application, where representative parameters include, but are not limited to: length, melting temperature (TM), non-homology with other regions of the genome, signal intensities, kinetic properties under hybridization conditions, and proximity to the capture site, etc. In certain embodiments, the oligonucleotides are selected so as to discriminate between species believed to be present in the community. Proximity between the capture site and the desired oligonucleotide sequence will influence the quantitative parameters of the assay, where oligonucleotides that are closer to the capture site, e.g. within around about 500 nucleotides, within around about 350 nucleotides, etc. are represented with greater efficiency compared to oligonucleotides that are selected at more distal sites. This difference in detection sensitivity may be influenced during upstream processing of the sample, where average shear fragment size determines the downstream detection. In this case, longer average fragment sizes generated during upstream shearing have a greater probability of including an oligonucleotide array probe sequence that is more distal to the capture site, and vice-versa.

Standard hybridization techniques (using high stringency hybridization and washing conditions) are used to assay a nucleic acid array. For a descriptions of techniques suitable for in situ hybridizations see, Gall et al. Meth. Enzymol., 21:470-480 (1981) and Angerer et al. in Genetic Engineering Principles and Methods Setlow and Hollaender, Eds. Vol 7, pgs 43-65 (plenum Press, New York 1985). See also U.S. Pat. Nos. 6,335,167; 6,197,501; 5,830,645; and 5,665,549; the disclosures of which are herein incorporate by reference.

In certain embodiments, highly stringent hybridization conditions may be employed. The term “highly stringent hybridization conditions” as used herein refers to conditions that are compatible to produce nucleic acid binding complexes on an array surface between complementary binding members, i.e., between immobilized features and complementary solution phase nucleic acids in a sample. Representative high stringency assay conditions that may be employed in these embodiments are provided above. The above hybridization step may include agitation of the immobilized features and the sample of solution phase nucleic acids, where the agitation may be accomplished using any convenient protocol, e.g., shaking, rotating, spinning, and the like. Following hybridization, the surface of immobilized nucleic acids is typically washed to remove unbound nucleic acids. Washing may be performed using any convenient washing protocol, where the washing conditions are typically stringent, as described above.

Following hybridization and washing, as described above, the hybridization of the labeled nucleic acids to the array is then detected using standard techniques so that the surface of immobilized features, e.g., array, is read. Reading of the resultant hybridized array may be accomplished by illuminating the array and reading the location and intensity of resulting fluorescence at each feature of the array to detect any binding complexes on the surface of the array. Other reading methods including other optical techniques (for example, detecting chemiluminescent or electroluminescent labels) or electrical techniques (where each feature is provided with an electrode to detect hybridization at that feature in a manner disclosed in U.S. Pat. No. 6,221,583 and elsewhere). In the case of indirect labeling, subsequent treatment of the array with the appropriate reagents may be employed to enable reading of the array. Some methods of detection, such as surface plasmon resonance, do not require any labeling of the nucleic acids, and are suitable for some embodiments.

Results from the reading or evaluating may be raw results (such as fluorescence intensity readings for each feature in one or more color channels) or may be processed results, such as obtained by subtracting a background measurement, or by rejecting a reading for a feature which is below a predetermined threshold and/or forming conclusions based on the pattern read from the array (such as whether or not a particular feature sequence may have been present in the sample, or whether or not a pattern indicates a particular condition of an organism from which the sample came).

### Utility

In some embodiments, the methods of the invention are used to identify microorganisms in complex samples.

Microbial symbionts form a prominent fraction of hitherto uncultured microorganisms. They live in continued close association with other organisms that can be categorized as obligate or facultative, mutualistic or parasitic, and ecto- or endosymbioses. Although many symbionts have been named solely on the basis of morphological criteria, their taxonomic position is essentially unknown. From a microbial perspective, the host organism can be regarded as a small ecosystem that is inhabited only by a few, often only one, well-adapted species. From a microbiologist's standpoint, such samples have a limited complexity and are ideally suited for CAPRA analysis.

Clinicians have long been aware of human diseases that are associated with visible but nonculturable microorganisms. For examples, the identification of slowly growing pathogens such as the members of the genus Mycobacterium are of great interest from a public health perspective. The same techniques may be used to analyze animal and plant pathogens.

Soils represent probably the most complex and the most difficult of environments to study. Microbial diversity often appears to be overwhelming, as demonstrated by the occurrence of several thousand independent genomes of standard soil bacterium complexity in one soil sample. The identification and monitoring of these species is of great interest from an environmental perspective, to ensure that organisms of interest are present where bioremediation, nitrogen fixation, etc. is to be performed.

Planktonic life as individual cells living in aqueous suspensions represents just one possible survival strategy of microorganisms. The second strategy is the colonization of solid surfaces or other interfaces by the formation of so-called biofilms. These immobilized consortia often catalyze important microbial transformations. Likely advantages of this lifestyle are the higher availability of nutrients on surfaces and the possibility of optimal long-term positioning in relation to other microorganisms or physicochemical gradients. Molecular and microscopic identification of defined bacterial populations in multispecies biofilms is of great interest.

### Kits

Also provided are kits for use in the subject invention, where such kits may comprise containers, each with one or more of the various reagents/compositions utilized in the methods, where such reagents/compositions typically at least include a collection of immobilized oligonucleotide features, e.g., one or more arrays of oligonucleotide features, and reagents employed in labeled nucleic acid production, e.g., random primers, buffers, the appropriate nucleotide triphosphates (e.g. dATP, dCTP, dGTP, dTTP), DNA polymerase, labeling reagents, e.g., labeled nucleotides, and the like.

Finally, the kits may further include instructions for using the kit components in the subject methods. The instructions may be printed on a substrate, such as paper or plastic, etc. As such, the instructions may be present in the kits as a package insert, in the labeling of the container of the kit or components thereof (i.e., associated with the packaging or sub-packaging) etc. In other embodiments, the instructions are present as an electronic storage data file present on a suitable computer readable storage medium, e.g., CD-ROM, diskette, etc.

The following examples are offered by way of illustration and not by way of limitation.

### EXPERIMENTAL

### Example 1

### Gene Sequence Capture and Random Amplification for Generating Clone Libraries

Cloning and sequencing of 16S rRNA genes from the environment represents a common approach to exploring microbial diversity. However, limitations with identifying conserved priming sites for PCR may underestimate diversity, since not all organisms share the so-called “universal” priming sites. In addition, the degree of phylogenetic resolution of the 16S rRNA gene means that downstream analyses of community composition are limited to the genus-species level. Provided herein is a novel method for bead-based sequence capture and random amplification of target genes, where the captured material is suitable for downstream cloning and sequencing applications. Success in the technique depends on efficient clearing of background genomic DNA in order to generate a high signal to noise ratio in the clones. Initial experiments with a pure culture of S. oneidensis demonstrate a capture efficiency of 1:6.

The microbial world is exceedingly diverse. Recent reports suggest that up to 13,000 different types of organisms comprise the microbiota of the human gut, and estimates of diversity in terrestrial ecosystems range from 4,000 to 10,000 different species per gram of soil. Traditional bacteriological techniques allow for only a fraction of this estimated diversity to be cultured. Instead, much of what is known about prokaryotic diversity is based on molecular methods of analysis. The study of conserved genes has revealed much about the disparity between the culturable and unculturable fractions of microorganisms. In most cases, part of the initial exploration of this diversity involves retrieval and analysis of the 16S ribosomal RNA genes as a first look into community composition and phylogeny.

The 16S rRNA gene has become the standard for studying microbial relatedness. The gene encoding the 16S ribosomal RNA is an important house-keeping gene that is present in at least one copy per cell, and has highly conserved sequences that are valuable priming sites for the Polymerase Chain Reaction. Researchers have also come to understand much about the mutational constraints of the structural 16S rRNA gene and have amassed large databases of sequence information that describe the variations in sequence across the prokaryotic domain. These databases offer insight for the development of group-specific PCR primers that allow for a narrower focus on a given subset of microorganisms. Thus, the combination of the 16S rRNA gene as a marker for phylogenetic studies and the PCR as a tool for amplifying sequences presents a compelling paradigm for community analysis.

Despite the advantages of the 16S rRNA gene for studying phylogeny, there are reasons to look elsewhere for genetic markers to identify and monitor microbial populations. One factor is the level of phylogenetic resolution of the 16S gene compared to other housekeeping genes. Protein-encoding genes are able to accumulate silent mutations because of the degeneracy of the amino acid code, allowing for nucleotide variations in the third or “wobble” position of the codon (as well as the first position for leucine, arginine, and serine.) The ribosomal RNA genes, in contrast, code for the structural components of the ribosome and mutations are limited to those that do not interfere with the proper folding of the rRNA. Unfortunately, the same mutational constraints that provide valuable PCR priming sequences are the same constraints that limit the accumulation of sequence differences between different microbial species and strains.

Another point to consider is the use of highly conserved sequences for PCR priming sites. What is termed “universal” in this case are short stretches of code that have similar and recognizable sequences across broad bacterial lineages, but do not necessarily have 100% sequence conservation. In the Comprehensive Microbial Resource database, for example, there are completely sequenced organisms with less than 65% sequence similarity to the so-called “universal” sites. Although these priming sequences have allowed for deeper investigations of poorly cultivable microorganisms, the question remains as to whether there are populations that are missed simply because they diverge from the most-commonly used PCR priming sites.

In addition to the considerations of genetic code resolution and priming sites, there is another factor that is of growing concern in the field of molecular microbial community analysis. This has to do with the observation that organisms can harbor multiple copies of the 16S rRNA gene and that the different copies within the organism can accumulate mutations that represent microheterogeneity in clone libraries. Microheterogeneity and differences in copy number between species also in uence the interpretation of other community analysis techniques. As the biases and limitations of the 16S gene become apparent, the question arises as to whether these limitations are surmountable. Although it is possible to clone fragments of entire genomes and study phylogeny without using PCR primers, metagenome analysis is not practical for most research budgets. PCR primers can be designed to target specific groups instead of broad-sweeping categories, but this limits the study of organisms to those that are already known to exist. And the problem of copy number bias may be addressed by applying correction factors to account for microheterogeneity and managing databases of organisms with known rrn copy number, but these represent only “patches” on a much larger problem.

In terms of phylogenetic resolution, several researchers have moved to studying conserved housekeeping genes that encode for proteins. These genes include those that encode for cell replication machinery, such as RNA and DNA polymerases, gyrases, and elongation factors. The advantage of these genes is that they are discriminatory at the species or strain levels and are found in a single copy per cell, but the limitation is that they are difficult to incorporate into a PCR protocol for a universal scale. That is, these genes may have amino acid sequences that are highly conserved, but the variability in the wobble positions of the nucleotide sequence makes it difficult or impossible to design PCR primer pairs for universal coverage.

The present invention provides a novel method for studying microbial diversity that uses bead-based gene capture for recovering genes of interest from the environment. In order to develop bead-based capture probes, various protein-encoding genes were analyzed for suitable DNA capture sites. The rpoC gene, which encodes for the β′ subunit of the DNA-dependent RNA polymerase, was discovered to have a short sequence of amino acids with 100% sequence conservation across all eubacteria and archaea known in the sequence databases. This amino acid sequence corresponds to the Mg-chelating center of the RNA polymerase enzyme. In addition, this strictly conserved region was found to lie immediately adjacent to more variable regions of sequence that allow for species and/or strain-level discrimination. For the capture probes, biotin-labeled oligonucleotides were synthesized that represented all possible combinations of the strictly conserved amino acid sequence. Affixing the biotin-labeled probes onto streptavidin-coated paramagnetic particles resulted in a type of molecular “fishhook” that was used to capture rpoC genes from diverse environments.

Although magnetic bead-capture protocols have been used to enrich genes of interest, one of the problems is that captured material represents only yecto- to atto-mole quantities of the desired genes. Thus, most bead-based gene capture protocols have only been used as simple precursors to traditional PCR amplification, where bead enrichment aids in the detection of genes that would otherwise be obscured by background. In order to overcome the sensitivity problem, a random PCR protocol was used to amplify copies of the genes that were enriched by bead-capture. In random amplification, fully degenerate hexamers are used to randomly prime and polymerize new strands of DNA without regard to sequence specificity in the original templates. By enriching the gene of interest on beads and removing sufficient background material, the majority of randomly amplified fragments should represent the intended target.

The following experiments exemplify the combination of bead-based sequence capture and random amplification for the purposes of cloning and sequencing novel rpoC genes from the environment. The term “CAPRA” was coined to reflect both the Capture and Random Amplification aspects of the technique. The success of CAPRA depends on the ability of the bead probes to capture the desired sequence, as well as the ability of the wash protocols to retain the captured sequences and eliminate background. Results with CAPRA demonstrate the capture and cloning of desired genes from the environment.

Materials and Methods

DNA samples and preparation. In order to test the ability of bead-based DNA capture probes to retrieve desired gene sequences, a pure culture of Shewanella oneidensis strain MR-1 was used as a control. Overnight cultures were extracted using either Bactozyme (Molecular Research Center, Cincinnati, Ohio) or MoBio DNA isolation kits (MoBio Laboratories, Inc., Carlsbad, Calif.) according to the kit protocols. For the MoBio protocol, the bead beating step was carried out using a BioSpec Products Mini Bead Beater at 5000 rpm for 60 seconds. Extracted DNA was diluted to a concentration of 50 ng/μl and sheared into randomly-sized fragments using a HydroShear apparatus (Genomic Solutions, Ann Arbor, Mich.). The HydroShear was set at Speed Code 12 in order to generate a range of fragments with an average length of 4000 base pairs. In addition to the control, bead capture and random amplification was performed on a mixed microbial community that was sampled from the aeration basin of the Palo Alto Water Quality Control Plant, Palo Alto, Calif. The mixed community was extracted using the MoBio Soil DNA isolation kit as described above. Recovered DNA was diluted to 50 ng/μl before shearing with the HydroShear at Speed Code 12.

Beads and probes. Streptavidin-coated MagneSphere paramagnetic particles were purchased from Promega in 0.6 ml aliquots and were used in a two-position MagneSphere Technology magnetic separation stand (Promega, Madison, Wis.) Oligonucleotide probes were synthesized with a 5′-biotin molecule and a polynucleotide A(12) linker with one of the following two sequences: a Shewanella-specific rpoC probe (5′-GACGGTGACCAAATGGC-3′); or a degenerate rpoC probe that accommodated all possible combinations of the amino acid sequence FDGDQMA (5′-TTYGAYGGNGAYCARATGGC-3′). Probes were reconstituted to a final concentration of 10 μM for the working stock, and 10 μl was used per capture reaction. According to the binding capacity of the paramagnetic particles as reported by Promega, 100 pM of biotin-labeled probe covers approximately 20% of the streptavidin binding surface in each 0.6 ml tube of particles.

Hybridization and wash protocols. The hybridization protocol was developed as a modification of a method published by Mangiapan et al. Unbound biotin probe was added directly to the DNA sample, then streptavidin-coated particles after a given hybridization period. A variety of hybridization and wash regimes were tested, and the combination giving the best capture and the least background was used throughout the study. The optimized protocol is as follows: 50 μl of genomic DNA (representing 1-4 μg of DNA) was heated to 95° C. in a water-filled heat block for 5 minutes then quenched in an ice slurry. Ten microliters of biotin-labeled probe was added to the cooled sample, followed by 450 μl of DIG EasyHyb buffer (Roche Applied Science, Indianapolis, Ind.) and the sample was transferred to a 37° C. incubator and rotated gently for 2 hours. To prevent leakage during the heating and hybridization steps, Safe-Lock tubes (Eppendorf, Westbury, N.Y.) were used for all samples.

Streptavidin-coated microspheres were prepared by washing three times with 2×SSC buffer, no earlier than 20 minutes prior to the conclusion of the hybridization period. The final wash buffer was removed immediately prior to the addition of sample. Samples were added to prepared beads and allowed to incubate with gentle rotation at 37° C. for 20 minutes. To remove background DNA, beads were drawn aside using a magnetic separation stand and the supernatant was removed. The beads and captured material were washed a total of 4 times, once with 300 μl of 2×SSC, and three times with 300 μl of 1×SSC. The best results were obtained when each wash step was allowed to incubate for 5 minutes with gentle rotation at room temperature. Captured DNA was eluted with 3 volumes of DNase-free water for a total of 400 μl. This material was concentrated using a Montage PCR Centrifugal Concentrator (Millipore, Billerica, Mass.) and recovered from the membrane filter in a volume of 20 μl of DNase-free water.

Quantitative-PCR analysis of signal to noise ratio. In order to gauge the efficiency of the gene capture and random amplification technique prior to cloning, a quantitative PCR protocol was developed to measure the ratio of rpoC and a gene selected at random to serve as a proxy for background noise, in this case, uridine kinase (udk). Shewanella-specific primer sequences for rpoC were located within 700 bases downstream of the capture site (forward primer, SonF: (SEQ ID NO:13) 5′-AACTATCTCTGGTGCCTCTGTCGGTATC-3′ and reverse primer, SonR: (SEQ ID NO:14) 5′-CGCACTTGCCCAGATGTCGATTACTTT-3′; for udk, the priming sequences were udkF: (SEQ ID NO:15) 5′-GACCATCCCAAAGCGTTAGA-3′ and udkR: (SEQ ID NO:16) 5′-ATTGCAGGAACATAGGACGG-3′.) Q-PCR reactions were run using an Applied Biosystems Model 7000 Real Time PCR cycler (Applied Biosystems, Foster City, Calif.) and ABI's SYBR Green Mastermix was used for amplification reactions where SYBR served as a general indicator of DNA amplification. The Q-PCR reaction protocol used a hot-start at 95° C. for 10 minutes, followed by 40 reaction cycles: 95° C. for 10s; 58° C. for 30s; and 72° C. for 30s.

Random amplification protocol A 2-step random amplification method was utilized. For the first reaction, 7 μl of bead-captured material was mixed with 2 μl 5× Sequenase buffer and 1 μl of 40 μmol/μl Primer A (SEQ ID NO:17) (5′-GTTTCCCAGTCACGATCNNNNNN-3′). This mixture was heated to 94° C. for 2 minutes, then cooled to 10° C. and held for 5 minutes in order to allow the primers to anneal. At this point, 5 μl of reaction buffer was added (1× Sequenase buffer, 300 uM each dNTP, 15 mM dithiothreitol, 150 μg/ml bovine serum albumin, and 0.8 U Sequenase enzyme). After adding the reagents, the temperature was ramped from 10 to 37° C. over 8 minutes and then held at 37° C. for an additional 8 minutes. This cycle was repeated a second time, except that 1.2 μl of a 1:4 dilution of Sequenase enzyme (0.9 μl Sequenase dilution buffer, 0.25 μl Sequenase enzyme) was added instead of reaction buffer. After the two cycles, the reaction mixture was diluted to 60 μl and used for Step 2.

The second step of the random amplification protocol more closely resembles a typical PCR reaction. In this case, the template from the first step was amplified with Primer B, which represents only the specific 5′ portion of Primer A (SEQ ID NO:18) (5′-GTTTCCCAGTCACGATC-3′). The reaction was carried out with 5-10 μl of template in a 50 μl mixture of 1× FailSafe Premix F (Epicentre Technologies, Madison, Wis.), 1 uM Primer B, and 1.25 U of Amplitaq DNA Polymerase LD (Applied Biosystems, Foster City, Calif.). The cycling conditions for this reaction were 30 seconds at 94° C., 30 seconds at 40° C., 30 seconds at 50° C. and 1 minute at 72° C., for a total of 30-35 cycles. The lowest cycle number that gave a visible smear using gel electrophoresis (from 650 to 1500 base pairs) was used for cloning.

Cloning and sequencing. During the development phase of the method, captured material was checked for its signal to noise ratio using Quantitative PCR, and samples that showed a ratio greater than 200:1 copies of rpoC to udk were used for cloning. An Invitrogen TOPO-TA Cloning Kit for Sequencing pCR4.0 (Invitrogen Corp., Carlsbad, Calif.) was used to generate clones, and 0.5 to 1 μl of PCR product was used per ligation reaction. The rapid transformation protocol was followed in order to prevent cell multiplication and the possibility of picking colonies that represented duplicates of the same insert. Transformed cells were plated on Luria Bertaini agar plates with 100 μg/ml ampicillin and 80 μg/ml X-Gal. All white colonies that appeared after overnight incubation were picked, transferred to LB broth with 50 μg/ml ampicillin, and sent for sequencing. The preparation of plasmid DNA and unidirectional sequencing was performed by MCLab, Inc. (South San Francisco, Calif.)

Results

Capture protocol. The strongest effect on signal to noise ratio was the stringency of the wash buffers and the length and degree of mechanical mixing during the washing steps. Five minute incubations with gentle rotary mixing worked better and more reproducibly than shorter and more vigorous mixing protocols (e.g. slow vortexing at speed 4, or gentle tapping of the tip of the tube.) The capture protocol also performed well using Roche DIG EasyHyb buffer at 370 C, compared to SSC buffers alone. The use of this hybridization regime reduced the dependence on hybridization temperature for assuring good capture.

Quantitative-PCR. Quantitative-PCR allowed for optimization of several factors involved in the capture protocol, including shear fragment length and hybridization and washing conditions. This was especially valuable, because captured fragments represented a quantity of DNA that fell below most means of detection. After capture, measured quantities of rpoC gene reflected a capture efficiency from 1% to 9%, depending on variations in the washing regime. The signal to noise ratio for Shewanella rpoC and udk genes ranged between 100 and 400, though the higher signal to noise ratios were accompanied by lower total recovery of rpoC. Despite the prevalence of rpoC compared to udk, it should be noted that udk represents only a small fraction of the 5.14 Mb of DNA that could serve as background. Thus, cloning was used as the ultimate measure of signal to noise ratio.

Random amplification. Shearing of the genomic DNA with the Hydroshear (speed code 12) produced a range of fragments with an average size of 3-4000 base pairs, and random priming and amplification further truncated this length to a range of 650-1300 bp and average of 900 base pairs. Sequenced inserts ranged in size from approximately 500 to 1100 base pairs with an average of 750 bp. However, 1 kb was the approximate upper length limit for quality reads in the sequencing reactions, and 25% of the clones had reached this limit.

Sequence analysis of cloned inserts. The sequenced inserts were trimmed of vector and checked for identity using NCBI Blast, and the ratio of rpoC fragments to non-rpoC fragments was scored. Both the Shewanella-specific capture probe and the universal degenerate capture probe were tested against a pure culture of Shewanella oneidensis, and the signal to noise ratio of the clones was approximately 1:6 and 1:7, respectively. That is, for every clone with an rpoC gene as insert, there were 6 to 7 clones that contained random genes from the background of the genome. In addition to undesired background genes, between 20% and 42% of sequenced inserts were represented by cloning vector. This was unexpected due to the presence of the lethal ccdB gene as a negative selector in the pCR4.0 plasmid of the TOPO-TA cloning kit.

This observation led to the use of blue-white screening in later cloning experiments. The inserts that represented rpoC or adjacent genes in Shewanella oneidensis were identified using NCBI Blast, and the numerical positions of each gene fragment were noted. Interestingly, the majority of inserts from the rpoBC operon were observed in the last colonies picked, and these represented the smallest and least well-isolated of the colonies. This observation led to a reversed colony-picking strategy, and later cloning experiments affirmed the notion that rpo inserts generate small colonies. The collection of sequenced rpo genes was plotted according to sequence position of Shewanella oneidensis, and this allowed for a graphical representation of gene coverage.

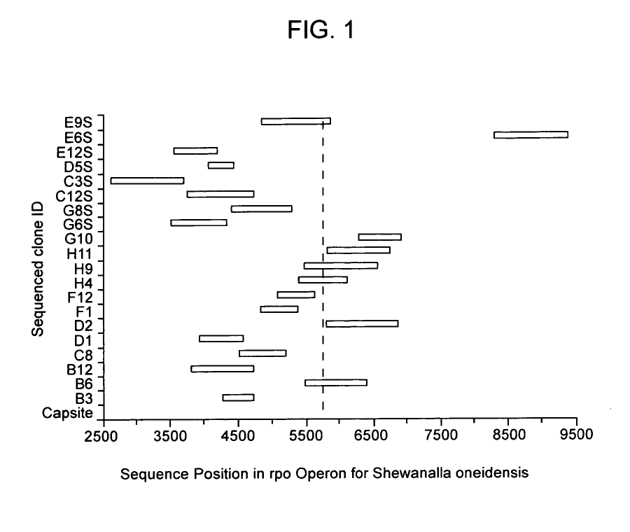

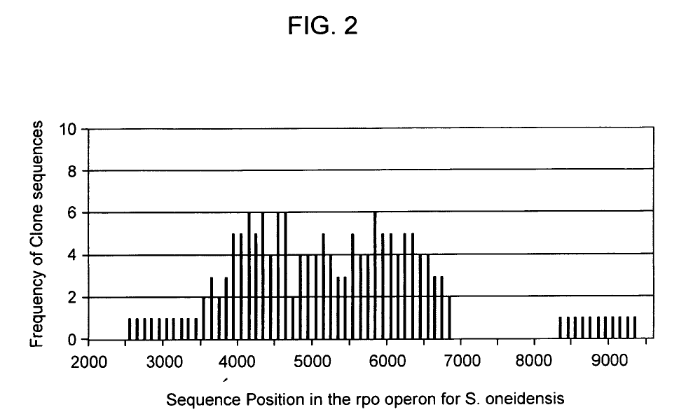

FIG. 1 shows the sequence coverage of 20 clones compared to the location of the capture site, and clones represented by an “S” suffix were obtained from the Shewanella-specific capture probe. Capture of gene sequences from the pure culture showed cumulative sequence coverage over a 4300 bp stretch of the rpo operon, although fragments seem to be localized upstream of the capture site. For the Shewanella-specific capture probe, for example, seven of eight fragment sequences lie upstream of the capture site. Frequency of coverage of any particular point was also determined in FIG. 2, and a minimum of 2 and maximum of 6 sequences cover the gene ranging from 2000 bases upstream and 1300 bases downstream of the capture site.

Sequences captured and cloned from the environmental sample were processed and analyzed in a similar manner as for the pure culture control. Cloning efficiency was observed to be low, with repeated cloning attempts yielding an average of approximately 20 white colonies per ligation reaction. Sequenced inserts were trimmed of vector and compared to the genomic databases using the nucleotide-to-protein function of NCBI Blast. In one cloning experiment, one positive rpoC insert was observed per 18 colonies picked, and a second experiment gave 2 rpoC-like inserts per 28 colonies. The identities of these sequences shared less than 72% amino acid sequence conservation with the most closely related organisms in the database, suggesting that the rpoC sequences captured were in fact novel.

The methodology of the present invention is independent of the 16S ribosomal RNA gene and the constraints of traditional 2-primer PCR. Bead-based oligonucleotide probes were used to capture the genes encoding the DNA-dependent RNA polymerase μ′ subunit, and initial results suggest that cloning and sequencing of captured inserts is indeed possible. At the present time, the optimized hybridization and wash protocol gives a signal to noise ratio of approximately 1:6 for S. oneidensis. The rpoC gene represents approximately 4 kb out of a possible 5 Mb in the Shewanella genome, thus this represents an enrichment factor of approximately 1250 times.

An alternative for screening clones is quantitative PCR and Taqman probe technology from Applied Biosystems, Inc (Foster City, Calif.). For this technique, a set of PCR primers are used to amplify an insert, and a fluorescent probe is designed that falls within the amplified product. As the Taq polymerase enzyme elongates from the priming sites, its 5′-3′ exonuclease activity cleaves apart any probe that may be bound on the target strand and generates a fluorescent signal. This type of Taqman assay may be modified for use in screening clones, where primers are used that target the conserved priming sites on the vector instead of the insert. A Taqman probe that targets a conserved region of rpoC would be able to hybridize to its target, and amplification from the vector priming sequences would generate a signal.

The ability to design different kinds of capture probes illustrates one of the strengths of the CAPRA technology. The concept of capture and random amplification does not depend on any particular sequence or gene, and may be applied to genes of varying degrees of conservation. For example, the CAPRA methodology may be used to study 16S rRNA genes, despite the level of resolution and the copy number biases. The ability to study 16S rRNA genes may prove to be a valuable transition, especially for environments that have been extensively studied using 16S rRNA-based community analysis tools.

### Example 2

### CAPRA: A Novel Method for Universal and Quantitative Assessment of Microbial Communities

The following experiments demonstrate a novel method for retrieving genetic information using paramagnetic beads and a strictly conserved set of eubacterial probes for the rpoC gene. Gene capture was combined with a random PCR amplification protocol, and results demonstrated that initial and final gene ratios were preserved. This combination of gene capture and random amplification suggests that representative and quantitative analyses are possible for complex mixtures of microorganisms.

PCR has been widely usec for the study of mixed communities. There are a number of factors that contribute to amplification bias, and these have to do with various interactions of primers and template. Several basic constraints that must be met for amplification of mixed templates: all molecules must be equally accessible, primer and template hybrids should form with equal efficiency, polymerization efficiency should be the same for all, and substrate exhaustion should affect all templates equally.

Of these considerations, primer and template interactions deserve much of the attention because priming sequences differ among groups of organisms. The efficiency of primer binding depends on nucleotide composition of the template priming site, G+C content, and various chemical and thermal parameters of the PCR reaction. The exponential increase in template copies also leads to potential error. Random events in the early cycles of the PCR can get exacerbated over successive cycles, leading to a situation known as “drift”. Another consequence of exponential amplification is that primer concentrations decrease as templates increase, providing a situation where complementary strands begin to compete with primer for template binding. This is another problem for quantitative analyses, since ratios of different templates may begin to converge as the number of template copies reaches a “plateau” after multiple rounds of amplification.

What is needed is a method that allows for the study of genes with a finer degree of phylogenetic resolution, while at the same time preserves the advantages of the polymerase chain reaction and eliminates sources of systematic error. DNA probes provide a solution to the limitations of 16S rRNA genes and traditional 2-primer PCR. By attaching a single-stranded DNA probe onto the surface of a super-paramagnetic particle, desired genes are captured from the environment and eliminate the background of the genome. This approach has the effect of enriching the gene of interest relative to background DNA, and works with a single oligonucleotide capture sequence rather than two priming sites as required in traditional two-primer PCR. Thus, design parameters for capture probes are much less stringent than for PCR primers: there is no need to account for varying melting temperatures between primer and template hybrids; there is no fear of forming heterodimers; and it is possible to incorporate much higher levels of degeneracy into capture probes.

The ability to design probes with higher degeneracy allows for capture of protein-encoding genes, since these sequences present greater variability in the wobble positions of the nucleic acid code. Several conserved housekeeping genes have been evaluated for use in fine-resolution phylogenetic analyses, including genes that encode for cellular transcription, translation and replication machinery.

For example, the gene that encodes for the beta subunit of the DNA-dependent RNA polymerase, rpoB, has been substituted for the 16S gene in DGGE analysis of marine ecosystems and the rpoC gene has been used to characterize the phylogeny of marine isolates. One of the benefits of such genes is that they are found as a single copy per cell, lending to greater accuracy in community analysis techniques. The rpoC gene offers an exciting possibility for bead-based DNA capture, because this gene has a short stretch of amino acids with strict sequence conservation across all eubacteria currently populating the DNA databases. In addition, the site is also strictly conserved in archaea and has sufficient residue substitutions to render unique eubacterial and archaeal probe sets. After accounting for the different possible combinations of nucleotides in the wobble position of the eubacterial sequence, this mixture represents a truly universal set of capture probes for this group of organisms.

Using a bead-based capture approach for studying mixed communities allows enrichment of the genes of interest, however, one of the limitations is that the copies of captured genes are too few to be visualized, sorted, or otherwise analyzed. Random amplification techniques are used to exponentially increase the mass of genomic material without regard to sequence identity. These approaches make use of fully degenerate oligomers that serve as primers in polymerase-based amplification reactions. One of the key questions regarding random amplification is whether these techniques are able to preserve the initial ratios of genetic material, thus providing an opportunity to explore mixed microbial communities in a quantitative fashion.

In order to test the capture recovery and amplification ratios of the CAPRA technique, a quantitative-PCR protocol was developed to measure the copy number of rpoC genes of pure cultures that were mixed in various combinations. The results of these analyses suggest that both capture and random amplification independently preserve initial gene ratios, and that the combination of these two techniques fulfills the conditions for a potentially universal and quantitative technique

Methods

Sample preparation. Organisms were selected from the Comprehensive Microbial Resource database to serve as members of constructed communities, and were selected based on local availability. The pure cultures represented organisms whose genomes are completely sequenced, including Agrobacterium tumefaciens, Deinococcus radiodurans, Mycobacterium tuberculosis, Shewanella oneidensis, and Vibrio cholera. Overnight cultures of each organism were extracted using MoBio DNA isolation kit (MoBio Laboratories, Carlsbad, Calif.) using a BioSpec Products Mini Bead Beater at 5000 rpm for 60 seconds (BioSpec Products, Inc., Bartlesville, Okla.). Extracted DNA was diluted to a concentration between 50 and 100 ng/μl and sheared into randomly-sized fragments using a HydroShear apparatus (Genomic Solutions). The HydroShear was set at Speed Code 12 in order to generate a range of fragments with an average length of 4000 base pairs.

Communities were mixed in various combinations by combining equal volumes of extracted DNA from each of the pure cultures. A two-member mixture was created with S. oneidensis and A. tumefaciens, and a 3 member community included these two organisms plus D. radiodurans. A four member community was also generated with S. oneidnesis, A. tumefaciens, M. tuberculosis, and V. cholera. No specific attempts were made to affect the genomic ratios of different organisms, although most combinations fell within the same order of magnitude in terms of abundance of initial rpoC copy number.

Materials and Methods

Beads and probes. Streptavidin-coated MagneSphere paramagnetic particles were obtained from Promega in 0.6 ml aliquots and were used in a MagneSphere Technology magnetic separation stand (Promega, Madison, Wis.) Oligonucleotide probes were synthesized with a 5′-biotin molecule and a polynucleotide A(12) linker with a degenerate nucleic acid sequence accommodating all possible combinations of the amino acid sequence FDGDQMA (5′-TTYGAYGGNGAYCARATGGC-3′). Probes were reconstituted to a final concentration of 10 μM for the working stock, and 10 μl was used per capture reaction.

Gene Capture. A hybridization protocol for bead-based gene capture was modified from a method by Mangiapan et al. (1996) J Clin Microbiol, 34(5), 1209-15. DNA was first extracted from pure cultures using a Bactozyme DNA isolation kit and sheared with a GeneMachines Hydroshear apparatus to generate fragments with an average size of 4 kb (FIG. 2.) In order to denature the DNA sample, 50 μl of genomic DNA (representing 1-4 μg of DNA) was heated to 95° C. in a heat block for 5 minutes then quenched in an ice slurry. Ten μl of biotin-labeled probe was added to the cooled sample, followed by 450 μl of DIG EasyHyb buffer (Roche Applied Science, Indianapolis, Ind.) and the sample was transferred to a 37° C. incubator and rotated gently for 2 hours.

Streptavidin-coated paramagnetic microspheres were prepared by washing three times with 2×SSC buffer, and the final wash was removed immediately prior to the addition of sample. The samples of DNA and bound probe were added to prepared beads and allowed to incubate with gentle rotation at 37° C. for 20 minutes. To remove background DNA, beads were drawn aside using a magnetic separation stand and the supernatant was removed. The beads and captured material were washed a total of 4 times, once with 300 μl of 2×SSC, and three times with 300 μl of 1×SSC. The best signal to noise ratios for rpoC relative to background were obtained when each wash step was allowed to incubate for 5 minutes with gentle rotation at room temperature. Captured DNA was eluted with 3 volumes of DNase-free water for a total of 400 μl. This material was concentrated using a Montage PCR Centrifugal Concentrator (Millipore, Billerica, Mass.) and recovered from the membrane filter in a volume of 20 μl of DNase-free water.

Random amplification. A 2-step random amplification protocol was adapted from Bohlander et al. (1992) Genomics, 13(4), 1322-4, in order to amplify the captured material]. For the first reaction, 7 μl of bead-captured material was mixed with 2 μl 5× Sequenase buffer and 1 μl of 40 pmol/μl Primer A (SEQ ID NO:19) (5′-GTTTCCCAGTCACGATCNNNNNN-3′). This mixture was heated to 94° C. for 2 minutes, then cooled to 10° C. and held for 5 minutes in order to allow the primers to anneal. At this point, 5 μl of reaction buffer was added (1× Sequenase buffer, 300 μM each dNTP, 15 mM dithiothreitol, 150 μg/ml bovine serum albumin, and 0.8 U Sequenase enzyme). After adding the reagents, the temperature was ramped from 10 to 37° C. over 8 minutes and then held at 37° C. for an additional 8 minutes. This cycle was repeated a second time, except that 1.2 μl of a 1:4 dilution of Sequenase enzyme (0.9 μl Sequenase dilution buffer, 0.25 μl Sequenase enzyme) was added instead of reaction buffer. After the two cycles, the reaction mixture was diluted to 60 μl and used for Step 2. The second step of the random amplification protocol more closely resembles a typical PCR reaction. In this case, the template from the first step was amplified with Primer B, which represents only the specific 5′ portion of Primer A (SEQ ID NO:20) (5′-GTTTCCCAGTCACGATC-3′). The reaction was carried out with 5 μl of template in a 50 μl mixture of 1× FailSafe Premix F (Epicentre Technologies, Madison, Wis.), 1 μM Primer B, and 1.25 U of Amplitaq DNA Polymerase LD (Applied Biosystems, Foster City, Calif.). The cycling conditions for this reaction were 30 seconds at 94° C., 30 seconds at 40° C., 30 seconds at 50° C. and 1 minute at 72° C., for a total of 30 cycles.

Quantitative-PCR and CAPRA. Real-time PCR was used to evaluate the quantitative ratios of both non-homologous and homologous genes during various phases of the CAPRA assay. Initial experiments were performed with a pure culture of Shewanella oneidensis as a positive control, where capture efficiency was determined by measuring the ratio of rpoC compared to the recovery of a random background gene, uridine kinase, udk. Five different organisms were used to study the efficiency of gene capture for a mixture of homologous genes, including Agrobacterium tumefaciens, Deinococcus radiodurans, Mycobacterium tuberculosis, Shewanella oneidensis, and Vibrio cholerae which served as the internal standard. Specific Q-PCR primers were initially designed for each organism that fell within 800 bases downstream of the capture site, and this distance was based on an average genomic DNA shear fragment size of approximately 4 kb. Primers were subsequently redesigned to narrow the proximity between the capture and detection site to 400 bp. In both cases, specificities of Q-PCR primers were determined by a factorial experiment with each pure culture and primer set, and cross amplification was observed to be negligible or an insignificant contribution to the Q-PCR signal within the ratios of organisms tested. Test communities were prepared with unspecified quantities of DNA from each organism, and were measured by Q-PCR to determine the initial numbers of the various rpoC genes.

Q-PCR reactions were prepared with 1×SYBR Green Mastermix (Applied Biosystems, Inc., CA), organism-specific primers, and 1 μl of product from the random amplification reaction. The Q-PCR cycling profile consisted of a 95° C. hold for 10 minutes, followed by 40 cycles of 95° C. for 15 s and 60° C. for 1 m. Standard curves were generated for each template and corresponding primer pair, and sampling error among triplicate Q-PCR measurements was estimated at 10%. In order to evaluate the percentage recovery of each gene, the measured quantities of rpoC from the different organisms were normalized to the internal standard to determine an initial ratio, Qᵢ. For example, the initial ratio for A. tumefaciens relative to V. cholera was expressed as Atuᵢ/V chᵢ. After each step of gene capture and random amplification, the quantities of rpoC genes for each organism were again measured and normalized to the internal standard to obtain a final ratio, Q_{f}, with the percent recovery was expressed as Q_{f}/Qᵢ.

TABLE 1
Primer Pairs used for Q-PCR analyses

| | |
|---|---|
| Organism (gene) | Primers |

| | |
|---|---|
| Agrobacterium | (SEQ ID NO:1) Forward: 5′-TCCAAGATCCATGAAACGACGCCT-3′ |
| tumefaciens (rpoC) | (SEQ ID NO:2) Reverse: 5′-TTGGTCATTTCCTGGTTGCAGGTG-3′ |
| Deinococcus | (SEQ ID NO:3) Forward: 5′-GTACTACACCAGCCGTGAGCGTAT-3′ |
| radiodurans (rpoC) | (SEQ ID NO:4) Reverse: 5′-TCTACGATACGGCGTTGTTCGCTG-3′ |
| Mycobacterium | (SEQ ID NO:5) Forward: 5′-GTACTACACCAGCCGTGAGCGTAT-3′ |
| tuberculosis (rpoC) | (SEQ ID NO:6) Reverse: 5′-TCTAGGATACGGCGTTGTTCGCTG-3′ |
| Shewanella | (SEQ ID NO:7) Forward: 5′-GTACTACACCAGCCGTGAGCGTAT-3′ |
| oneidensis (rpoC) | (SEQ ID NO:8) Reverse: 5′-TCTACGATACGGCGTTGTTCGCTG-3′ |
| Shewanella | (SEQ ID NO:9) Forward: 5′-GACCATCCGAAAGCGTTAGA-3′ |
| oneidensis (udk) | (SEQ ID NO:10) Reverse: 5′-ATTGCAGGAACATAGGACGG-3′ |
| Vibrio | (SEQ ID NO:11) Forward: 5′-CCAACGGTCGTGTCAATCATCTTG-3′ |
| cholera (rpoC) | (SEQ ID NO:12) Reverse: 5′-AAGGCGAAGGTATGTACCTGACTG-3′ |

Results

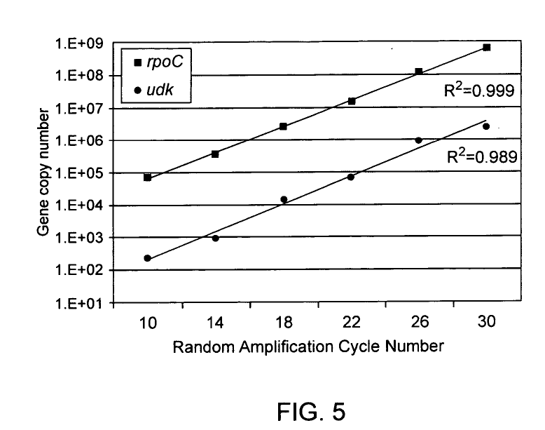

Optimization of the CAPRA Assay. In order to develop the methodology, gene capture and random amplification was performed with a pure culture of Shewanella oneidensis as a positive control. Conditions were optimized by comparing the enrichment of the rpoC gene relative to a single-copy background gene selected at random, uridine kinase (udk). Given that these two genes are initially present in the S. oneidensis genome at a ratio of 1:1, the efficiency of capture was expressed as a signal to noise ratio of rpoC:udk. Under gentle wash conditions, the enrichment of rpoC was reproducibly observed by a factor of over 300 times compared to udk. After enrichment of rpoC, the random amplification of these non-homologous genes was observed to occur at an equivalent rate, indicating that there was no primer preference for either gene (FIG. 5.)

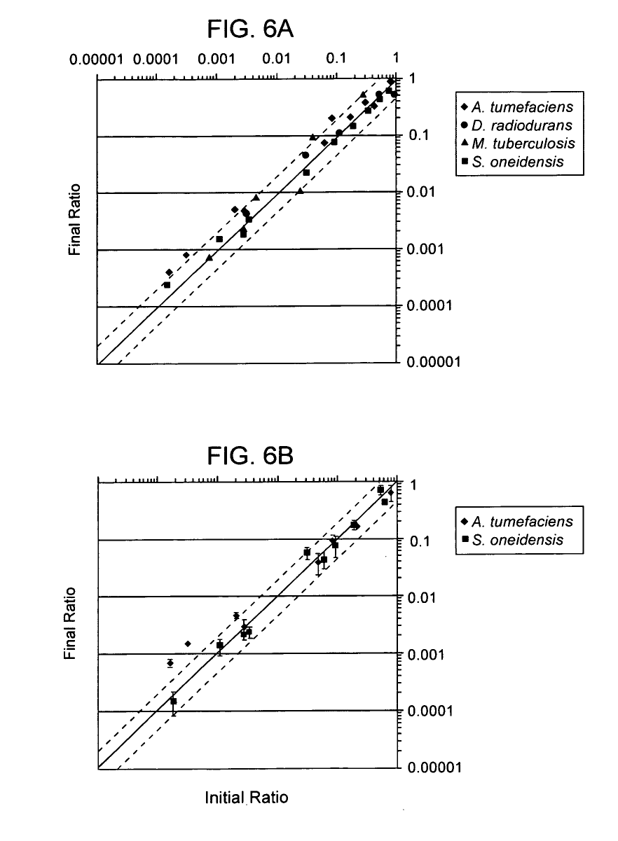

CAPRA for Homologous Genes. Measuring and discriminating homologous rpoC genes from a mixture of different organisms presented a challenge because of the sequence conservation within this gene and the constraints in primer design for the Q-PCR analytical technique. The results of gene capture for the mix of 5 organisms showed that ratios were preserved within a factor of two (FIG. 6a), even though the initial concentrations of rpoC genes differed from the internal standard by up to four orders of magnitude. After gene capture, samples were divided into 3 independent replicates and amplified in two steps using a random hexanucleotide PCR protocol. Aliquot samples were sacrificed after successive rounds of random amplification and ratios of the different organisms were measured using Q-PCR.

Amplification of rpoC genes from the V. cholerae standard showed exponential growth and strong agreement among replicates, but measurements for the other organisms were unexpectedly low and replicate samples diverged up to an order of magnitude. This degree of variation ran counter to the observations with random amplification of non-homologous genes, so it seemed unlikely that concentration differences between the organisms should be a factor. A review of the Q-PCR primer design suggested a possible explanation: primers for V. cholerae were located within 400 base pairs of the capture site, whereas the ideal Q-PCR priming sites for the other organisms were located up to an additional 400 bases downstream. Considering that random amplification produces a truncated set of amplicons relative to the initial fragment sizes, it seemed likely that Q-PCR priming sites distal to the capture site were being lost or disrupted during the random amplification step. After redesigning Q-PCR primer pairs for A. tumefaciens and S. oneidensis to narrow the proximity between the capture and priming sites to 400 bp, amplification of the rpoC genes from these organisms matched the rate for V. cholerae. However, redesigned primers for M. tuberculosis were not suitable under the standard Q-PCR conditions, and primers for D. radiodurans were not identified; thus, it was 3 of the 5 organisms in the mixed sample were accurately measured after random amplification.

The full CAPRA assay was evaluated for A. tumefaciens and S. oneidensis with V. cholerae as the internal standard, and the ratios of rpoC genes and the percent recoveries, Q_{f}/Qᵢ, were calculated as before. Again, the product ratios reflected the initial template ratios despite orders of magnitude differences in the concentrations of homologous genes (FIG. 6b.) Interestingly, the ratios remained preserved within a factor of two whether the organisms were present at nearly 1:1 or 1:10,000 relative to the standard. This suggests that deviation in the quantitative measurements is independent of the template ratios of the different genes. As in conventional PCR, the effects of these types of error can be dampened by combining replicate reactions. Assuming that quantitative measurements are independent of concentration and that all CAPRA experiments behaved as replicates for a given organism, the average percent recovery for S. oneidensis was 98±22% (95% confidence interval.) For A. tumefaciens, two samples corresponding to the lowest concentrations of rpoC genes (approximately 10 copies per μl after random amplification) differed from expected results by a factor of 4. Excluding these two points and averaging the ratio measurements for the remaining A. tumefaciens samples gave an average recovery of 97±39% (95% confidence interval). These results demonstrate that quantitative ratios are accurately preserved despite differences of several orders of magnitude between the test populations and the standard. Thus, the CAPRA assay offers compelling new opportunities for the study of microbial community dynamics and the roles of minority populations.

Gene capture and random amplification provides a useful strategy for universal and quantitative analysis of mixed microbial communities. The methods were demonstrated with the DNA-dependent RNA polymerase gene as a universal target, and can be applied to any target in any system. This allows for the identification and monitoring of genes ranging from the ubiquitous housekeeping genes to those that encode for more highly specialized functions. In addition to microbial diagnostics, the CAPRA assay can be used to identify and classify genotypes in higher organisms. These tools will become readily available as the CAPRA assay is coupled to suitable downstream detection technologies such as clone libraries and DNA microarrays.

Gene capture coupled to cloning has particular application in the discovery of novel microbial genotypes that are elusive in conventional PCR-based cloning assays. Genbank currently lists an impressive number of entries, but these represent less than 1% of the estimated 4 million different taxa in marine waters and 6 million taxa suggested for terrestrial environments. A recent report on the status of the microbial census suggests that current methods may not be adequate to sample this enormous diversity; the CAPRA assay provides a means to fill this need. Experiments in this laboratory with CAPRA and cloning demonstrate that novel sequences can be identified from environmental samples.

The CAPRA assay can also be combined with DNA microarray hybridization technology for sensitive and highly-multiplexed discrimination of captured fragments. PCR and random amplification protocols have already been used to increase the detection sensitivity in DNA microarray diagnostics, and results suggest that random PCR approaches introduce considerably less amplification bias for whole genomes compared to conventional PCR. The addition of gene capture as an enrichment step, as the results of this paper demonstrate, allows for gene selectivity and further enhances the sensitive and quantitative power of microarray detection by reducing the interference of background genomic DNA. At the same time, refinements and automation of the CAPRA technique can also help reduce various types of non-systematic error, leading to greater accuracy and precision in the measurements. Further experimentation will also help determine the full range of detection sensitivity relative to an internal standard as well as the absolute detection limits for any given target.

In an era of rapidly emerging applications in biotechnology, gene capture and random amplification represents a major step forward in identifying and monitoring organisms. The CAPRA assay offers several advantages for selectively and quantitatively amplifying multiple loci in a single reaction without prior knowledge of PCR priming sites. This ability to retrieve and amplify sequences in an unbiased manner has broad implications for developing accurate, universal, and quantitative tools that can be used to finely discriminate organisms across all known domains of life.

All publications and patent applications cited in this specification are herein incorporated by reference as if each individual publication or patent application were specifically and individually indicated to be incorporated by reference. The citation of any publication is for its disclosure prior to the filing date and should not be construed as an admission that the present invention is not entitled to antedate such publication by virtue of prior invention.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

What is claimed:

1. A method for polynucleotide characterization, the method comprising:
obtaining genomic DNA from a microbial sample;
contacting said DNA with a capture probe under hybridization conditions;
selecting for DNA bound to said capture probe;
randomly amplifying captured DNA;
characterizing genetic sequences in said randomly amplified population.

2. The method according to claim 1, further comprising the step of cloning said randomly amplified population in a replicable vector.

3. The method according to claim 2, further comprising sequencing inserts in said replicable vector.

4. The method according to claim 3, further comprising preparing a microarray comprising diverse genetic sequences obtained by said sequencing.

5. A microarray prepared by the method of claim 4.

6. The method of claim 1, further comprising hybridizing said sample to a microarray comprising a plurality of probes specific for diverse microbial coding sequences.
